(19) United States
(12) Patent Application Publication
(10) Pub. No.: US 2009/0198084 A1
(43) Pub. Date: Aug. 6, 2009

(54) PROCESS FOR THE DEPROTECTION OF PROTECTED AMINES

(76) Inventors: Paulus Lambertus Alsters, Maastrich (NL); Jorge Merijn Mathieu Verkade, Nijmegen (NL); Peter Jan Leonard Mario Quaedflieg, Elsloo (NL); Floris Petrus Johannes Theodorus Rutjes, Wijchen (NL)

Correspondence Address: NIXON & VANDERHYE, PC, 901 NORTH GLEBE ROAD, 11TH FLOOR, ARLINGTON, VA, 22203, US

(21) Appl. No.: 12/299,039

(22) PCT Filed: May 1, 2007
(86) PCT No.: PCT/EP2007/003833

§ 371 Date: Feb. 4, 2009

(30) Foreign Application Priority Data
May 2, 2006 (EP) EP06113390
Jul. 21, 2006 (EP) EP06117658

(51) Int. Cl. C07C 51/16; C07C 209/68

(52) U.S. Cl. 562/409; 564/384

(57) ABSTRACT

The present invention relates to a process for the deprotection of protected amine compounds, wherein the protected amine compound is contacted with an electrophilic oxidating agent that is optionally formed in situ, said electrophilic oxidating agent being selected from the group of I₂, Br₂, Cl₂ and compounds comprising a halogen atom having a formal oxidation state of 1+, 3+, 5+ or 7+, provided that the electrophilic oxidating agent is not iodobenzene diacetate. The process can be conveniently employed in the synthesis of 1,3-amino alcohols, β-amino acids and heterocyclic compounds, in particular β-amino acids.

The present invention relates to a process for the deprotection of protected amine compounds, in particular protected amine compounds that are N-protected by an aromatic group. The process according to the invention is for example very useful in the preparation of β-amino acids and 1,3-aminoalcohols based on asymmetric Mannich reactions where cleavage of the N-aryl group is required.

Mannich reactions and in particular asymmetric Mannich reactions are well known in the art. In these reactions substituted and optionally activated imines are used as the amino-group providing synthon, wherein the substituents may also act as a protective group for the amino function in subsequent transformations. Today, popular protective groups include phenyl groups substituted with one or more electron-donating groups such as alkoxy groups which are inter alia employed in (asymmetric) reduction of protected imines, three-component reactions to form protected amino acid amides and asymmetric Diels-Alder reactions. In particular, when N-p-methoxyphenyl protected imines are used in an asymmetric Mannich reaction, the enantioselectivity and diastereoselectivity are very high. However, the known methods for deprotecting such protected amines involve oxidative cleavage of the appropriate nitrogen-carbon bond and require large amounts of often environmentally unfriendly reagents and relatively complex work-up procedures. Moreover, the known methods provide low to moderate yields.

A well-known reagent for the oxidative cleavage of the amine-carbon bond in optionally substituted N-phenyl-protected amine compounds is cerium ammonium nitrate. However, 4-5 molar equivalents have to be used and yields are generally not higher than about 60%.

Porter et al., J. Am. Chem. Soc. 123, 10409-10410, 2001, discloses the asymmetric Mannich reaction of optionally substituted aliphatic aldehydes and o-methoxyphenyl amine and the subsequent removal of the o-methoxyphenyl group in the end-product by oxidative cleavage of the appropriate nitrogen-carbon bond under acidic conditions by using iodobenzene diacetate as the oxidant. However, also this method requires 4-5 molar equivalents of the unsafe and expensive oxidant and provides only moderate yields.

Ibrahem et al, Angew. Chem. Int. Ed. Engl. 43, 6528-6531, 2004, discloses an asymmetric Mannich reaction of ketones and p-methoxyphenyl amine followed by removal of the p-methoxyphenyl group by using iodobenzene diacetate.

De Lamo Marin et al., J. Org. Chem. 70, 10592-10595, 2005 discloses the deprotection of p-methoxyphenyl amines by anodic oxidation. However, this method, for large-scale production, requires special equipment and significant investments.

Consequently, there is a need in the art for a more efficient process for deprotecting protected amine compounds, in particular protected amine compounds that are N-protected with an aromatic group, that can be broadly applied, said process employing cheap and environmentally friendly reagents and proceeding with beneficial atom economy.

The present invention relates to a process for the deprotection of protected amine compounds, wherein the protected amine compound is contacted with an electrophilic oxidating agent that is optionally formed in situ, said electrophilic oxidating agent being selected from the group of 12, Br₂, Cl₂ and compounds comprising a halogen atom having a formal oxidation state of 1+, 3+, 5+ or 7+, provided that the electrophilic oxidating agent is not iodobenzene diacetate. The present invention further relates to a process for the asymmetric synthesis of amines and to the use of the electrophilic oxidating agent in the asymmetric synthesis of amines.

In this description and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

Where in this document the abbreviation PMP is used, it denotes the p-methoxyphenyl group. In this document, the terms “oxidating agent” and “oxidation agent” and “oxidising agent” are used interchangeably.

The present invention relates to a process for the deprotection of protected amine compounds, wherein the protected amine compound is contacted with an electrophilic oxidating agent that is optionally formed in situ, said electrophilic oxidating agent being selected from the group of 12, Br₂, Cl₂ and compounds comprising a halogen atom having a formal oxidation state of 1+, 3+, 5+ or 7+, provided that the electrophilic oxidating agent is not iodobenzene diacetate.

It is known that halogen containing compounds, such as for example N-chlorosuccinimide or trichloroisocyanuric acid, can be used in halogenation reactions. In particular electron rich aromatic compounds are easily halogenated. Such reactions are for example known from Journal of Medicinal Chemistry, 47(19), 4741-4754, 2004, and Journal of the Brazilian Chemical Society, 16(4), 695-698, 2005. Despite the known halogenation reactions, it surprisingly now has been found that halogen atom containing electrophilic oxidating agents selected from the group of 12, Br₂, Cl₂ and compounds comprising a halogen atom having a formal oxidation state of 1+, 3+, 5+ or 7+ are suitable to deprotect protected amine compounds.

Suitable electrophilic oxidating agents include for example trichloroisocyanuric acid (TCCA), N-chlorosuccinimide, dichloroisocyanuric acid, dichloroisocyanuric acid sodium salt, dichloroisocyanuric acid potassium salt, 1,3-dichloro-5,5-dimethylhydantoin, N-chloro-p-benzenesulfonamide sodium salt (Chloramine B), N-chloro-p-toluenesulfonamide sodium salt (Chloramine T), N,N-dichloro-p-toluenesulfonamide (Dichloramine T), N-chlorophtalimide, trichloromelamine, 1,3-dibromo-5,5-dimethylhydantoin, N-bromosuccinimide, N-bromoacetamide, N-bromocaprolactam, N-bromophthalimide, N-iodosuccinimide, 3-diiodo-5,5-dimethylhydantoin, periodic acid or a salt thereof, N-chlorosuccinimide, Dess-Martin reagents, hypochlorous acid or a salt thereof, pyridium bromide perbromide, bromine and iodine.

Mixtures of electrophilic oxidating agents according to the invention may also be used for the deprotection process according to the invention.

Halogen containing salts that may be used as electrophilic oxidating agents in the process according to the invention are for example sodium hypochlorite, lithium hypochlorite, potassium hypochlorite, calcium hypochlorite, sodium chlorite, sodium chlorate, potassium chlorate, magnesium chlorate, sodium hypobromite, sodium bromite, sodium bromate, lithium bromate, potassium bromate, magnesium bromate, sodium iodate, lithium iodate, potassium iodate, magnesium iodate, calcium iodate, sodium periodate, lithium periodate and potassium periodate.

According to the invention, the halogen atom has preferably a formal oxidation state of 1+, 5+ or 7+, most preferably a formal oxidation state of 1+. Preferred examples of the electrophilic oxidating agent are periodic acid or a salt thereof, hypochlorous acid or a salt thereof, trichloroisocyanuric acid, N-iodosuccinimide, N-bromosuccinimide and N-chlorosuccinimide. Most preferred are periodic acid or a salt thereof and trichloroisocyanuric acid.

It is furthermore preferred that the halogen atom is selected from the group consisting of chlorine, bromine or iodine. Most preferably, the halogen atom is chlorine or iodine. Accordingly, the more preferred examples of the electrophilic oxidating agent are periodic acid or a salt thereof, hypochlorous acid or a salt thereof, trichloroisocyanuric acid, N-iodosuccinimide, N-bromosuccinimide and N-chlorosuccinimide, whereas the most preferred examples of the electrophilic oxidating agent are periodic acid or a salt thereof, hypochlorous acid or a salt thereof, trichloroisocyanuric acid and N-chlorosuccinimide.

The electrophilic oxidating agent can be inorganic or organic. A preferred inorganic electrophilic oxidating agent is periodic acid or a salt thereof. Preferred organic electrophilic oxidating agents are trichloroisocyanuric acid, N-bromosuccinimide and N-chlorosuccinimide, most preferably trichloroisocyanuric acid. Most preferably, the electrophilic oxidating agent is an organic electrophilic oxidation agent.

It should be noted that the prior art processes for deprotection wherein cerium ammonium nitrate (CAN) is used as the deprotection agent, require a laborious work up procedure. However, it is an advantage of the process according to the invention, that after deprotection of the amine protected compound according to the invention, a practical and straightforward work up is possible. A possible work up procedure is described in the examples.

Although the process according to the invention can be conducted at any pH, it is preferred that the process for protected amines that are N-protected with an aromatic group is conducted at acidic pH.

The process according to the invention is preferably carried out at a pH of 7 or lower, more preferably 5 or lower, even more preferably 3 or lower. Lower pH values are especially preferred where the protected amine compound has functional groups that may give rise to epimerisation (or racemisation if only one chiral centre is present) of optically active centres, e.g. when the protected amine compound is a protected amino alcohol, in particular a 1,3-amino alcohol, or a protected β-amino acid. The lower limit of the pH is not extremely relevant, but for common practice it will not be lower than −1. The pH can be controlled by addition of an inorganic acid, an organic acid, or mixtures thereof in the appropriate amounts, wherein the acids are preferably protic acids.

The process according to the invention can be conducted with any protected amine compound, i.e. a compound comprising a secondary or a tertiary amine. The temperature may vary between wide limits, for example between 5° C. and 200° C. Preferably the process according to the invention is carried out at a temperature higher than 15° C., more preferably higher than 30° C. In this text ambient temperature is considered any temperature between 10° C. and about 40° C. However, if the process is performed at a temperature between about 10° C. and about 40° C., it is preferred that the protected amine compound is a compound comprising a secondary amine. If the protected amine compound is a tertiary amine, it can still be deprotected at a temperature between about 10° C. and about 40° C., although at a lower rate. However, the process proceeds more effectively if the process is conducted at elevated temperatures, preferably in the range of 30° C. to 200° C., more preferably in the range of 40° C. to 170° C., and most preferably in the range of 50° C. to 150° C. So in general it is preferred that the protected amine compound has the structure R—NH—PG, wherein the R—NH moiety is derived from the amine compound and the PG-moiety represents the protective group, e.g. an aryl group, in particular when the protected amine compound is labile at elevated temperatures.

The pressure at which the process according to the invention may be carried out may is not critical and may vary between wide limits, for example 0.01 bar and 100 bar.

Typically, the process according to the invention is carried out under atmospheric pressure.

The amount of the electrophilic oxidation agent, relative to the protected amine compound, is not critical. The molar amounts of the electrophilic oxidation agent are also dependent on the number of halogen atoms included in the electrophilic oxidation agent. In general, it is preferred to employ at least 0.2 equivalent of electrophilic oxidation agent per mol protected amine compound, more preferably at least 0.4 equivalent. Typically not more than 5.0 mol, equivalent of electrophilic oxidation agent per mol protected amine compound is used, preferably not more than 2.5 per mol protected amine compound. To give a good yield, periodic acid may often be used in about 1 equivalent of electrophilic oxidation agent per mol protected amine compound, while TCCA may be used in about 0.5 mol equivalent.

Typically, the process according to the invention is carried out in solution. Any different solvents may be used, including but not limited to all solvents mentioned in the examples. Also mixtures of solvents may be used. Preferably, the process according to the invention is carried out in the presence of water. Typically, the process is carried out in a mixture of solvents including water.

According to the invention, the amino group of the protected amine compound is preferably protected by an optionally substituted aryl or benzyl group, most preferably by an optionally substituted C₆-C₁₄ aryl group. Suitable examples of such optionally substituted aryl groups include optionally substituted phenyl and naphthyl groups. Additionally, it is preferred that such aryl or benzyl groups are activated by electron-donating groups, wherein the aryl or benzyl group is preferably substituted at positions that maximise the activating effect of the electron-donating substituent. Such substitution patterns are well known to the person skilled in the art and include for example the ortho- and para-positions of a phenyl ring. Consequently, it is preferred according to the invention that the amino group of the protected amine compound is protected with a phenyl group that is substituted with an electron-donating substituent, wherein the phenyl group is substituted at an ortho-position and/or the para-position. Obviously, the phenyl group may be substituted with the same or with different electron-donating groups. More preferably, the phenyl group is substituted with two substituents wherein one substituent is most preferably at the para-position. However, most preferably, the phenyl group is only substituted in the para-position.

In a preferred embodiment, the protecting group is a p-methoxyphenyl group (PMP).

Preferably, the electron-donating group has a Hammett σₚ-constant of less than −0.14. Examples for suitable electron-donating groups are methyl (σₚ=−0.14), t-butyl (σₚ=−0.15) and methoxy (σₚ=−0.28). The values for up are taken from J. March, Advanced Organic Chemistry, 4th Ed., page 280 (Table 9.4), 1992. Preferred electron-donating groups include linear or branched C₁-C₆ alkyl groups, cyclic C₃-C₆ alkyl groups, linear or branched C₁-C₆ alkoxy groups, cyclic C₃-C₆ alkoxy groups, linear or branched C₁-C₆ alkylthio groups, cyclic C₃-C₆ alkylthio groups and OH. More preferred electron-donating groups are linear or branched C₁-C₆ alkyl groups, cyclic C₃-C₆ alkyl groups, linear or branched C₁-C₆ alkoxy groups and cyclic C₃-C₆ alkoxy groups. Even more preferred electron-donating groups are linear or branched C₁-C₆ alkoxy groups and cyclic C₃-C₆ alkoxy groups. Yet even more preferred electron-donating groups are linear or branched C₁-C₆ alkoxy groups, even yet more preferably linear or branched C₁-C₄ alkoxy groups. Most preferably, the electron-donating group is methoxy. If the phenyl group has more than one substituent, their combined electron-donating capability is preferably equivalent to a Hammett σₚ-constant of less than −0.14. It is well known to the person skilled in the art which combinations of substituents provide such an electron-donating capability.

The present invention also relates to a process for the asymmetric synthesis of amines, said process comprising the steps of:
(a) reacting a carbonyl compound with an amine compound that is at least substituted by an aryl or benzyl group, preferably by a C₆-C₁₄ aryl group, under formation of a protected imine compound;
(b) converting the protected imine compound into a protected amine compound; and
(c) deprotecting the protected amine compound according to the process described above.

More in particular, the present invention relates to a process for the asymmetric synthesis of amines, said process comprising the steps of:
(i) reacting a first carbonyl compound with an amine compound that is at least substituted by an aryl or benzyl group, preferably by a C₆-C₁₄ aryl group under formation of a protected imine compound;
(ii) reacting a second carbonyl compound with the protected imine compound under formation of a protected amine compound; and
(iii) deprotecting the protected amine compound according to the process described above.

Step (b) and step (ii) of the amine synthesis process may be a Mannich reaction, optionally an asymmetric Mannich reaction, which is optionally performed in the presence of a catalyst. Steps (b) and (ii) may further comprise additional reactions steps which are conducted prior to steps (c) and (iii), e.g. a reduction of the keto functionality. Mannich reaction are well known in the art. The skilled person knows or can easily identify the suitable reactions conditions for carrying out the process for asymmetric synthesis of amines, in particular when using Mannich reactions

Preferably, the carbonyl compound is a ketone, an aldehyde, a protected ketone, a protected aldehyde or a precursor of a ketone or an aldehyde. It is furthermore preferred that the carbonyl compound is a prochiral carbonyl compound. The carbonyl compound may further comprise one or more, optionally protected, functional groups such as OH-groups, SH-groups, carboxyl groups, ester groups and the like. A preferred class of carbonyl compounds are those which have one or more OH-groups.

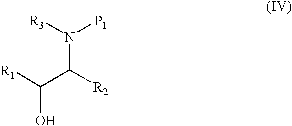

In case the protected amine contains an unprotected OH-group at the β-position, such as shown in formula (IV), submission to oxidation by the electrophilic oxidating agents of the present invention may result in occurrence of side reactions due to (partial) cleavage of the C—C bond of the 1,2-amino-alcohol moiety. In formula (0), R₁ and R₂ are each independently selected from the group of linear or branched, optionally substituted C₁-C₆ alkyl, alkenyl or alkynyl groups or from the group of optionally substituted C₄-C₁₂ cycloalkyl groups which optionally have one or more unsaturated, exocyclic or endocyclic, carbon carbon bonds or from the group consisting of optionally substituted C₆-C₁₂ aryl groups and optionally substituted C₇-C₁₂ aralkyl groups; R₃ is selected from the group consisting of hydrogen, linear or branched C₁-C₆ alkyl groups and C₄-C₁₂ cycloalkyl groups; and P1 is a protective group, preferably selected from the group consisting of the optionally substituted C₆-C₁₄ aryl groups that are preferably activated by electron-donating groups as described above.

If substituted, the alkyl, alkenyl, alkynyl, cycloalkyl, aryl and aralkyl groups may be substituted with optionally protected, functional groups comprising one or more heteroatoms such as oxygen, sulphur and nitrogen. Additionally, the alkyl, alkenyl and alkynyl groups may be interrupted by one or more heteroatoms such as oxygen, sulphur and nitrogen, whereas the cycloalkyl, aryl and aralkyl groups may comprise within their ring system one or more heteroatoms such as oxygen, sulphur and nitrogen.

Above mentioned side reactions, occurring during the deprotection of the amine protecting group in a compound of the formula (IV) by an electrophilic oxidating agent of the present invention, can be partially or fully avoided by first protecting the OH-group at the α-position, then performing the amine deprotection with an electrophilic oxidating agent of the present invention, and finally removing the OH-protecting group. For instance, the OH-group can be protected with a benzyl group, using benzylbromide and sodium hydride, followed by amine deprotection with trichloroisocyanuric acid or periodic acid—with limited or no side reactions due to cleavage of the C—C bond of the 1,2-amino-alcohol moiety—and finally removal of the benzyl group using hydrogenation with hydrogen and Pd/C under acidic conditions, giving the desired deprotected 1,2-amino-alcohol in high yield. The OH-group can, for example, also be protected with a tert-butyl-diphenylsilyl group, using tert-butyl-diphenylsilylchloride in the presence of imidazole, followed by amine deprotection with trichloroisocyanuric acid or periodic acid—with limited or no side reactions due to cleavage of the C—C bond of the 1,2-amino-alcohol moiety—and finally removal of the tert-butyl-diphenylsilyl group by HF/pyridine, giving the desired deprotected 1,2-amino-alcohol in high yield.

According to the invention, the amine that is prepared in the amine synthesis process is preferably selected from the group consisting of 1,3-amino alcohols, β-amino acids and heterocyclic compounds. β-Amino acids are preferably prepared in a one-pot synthesis, wherein as starting material the protected amine compound is employed and wherein the deprotected amine compound is converted into the corresponding β-amino acid. This last oxidation step is preferably catalysed by a catalyst or catalyst system.

Accordingly, the present invention also provides a method for the preparation of β-amino acids comprising the steps of:

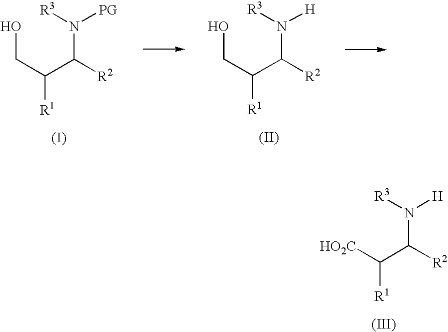

(A) deprotecting a protected amine compound according to formula (I) by contacting said protected amine compound with an electrophilic oxidating agent that is optionally formed in situ, said electrophilic oxidating agent being selected from the group of I₂, Br₂, Cl₂ and compounds comprising a halogen atom having a formal oxidation state of 1+, 3+, 5+ or 7+ preferably with the proviso that the electrophilic oxidating agent is not iodobenzene diacetate, to a deprotected amine compound according to formula (II); and
(B) oxidating the deprotected amine compound according to formula (II) to a β-amino acid according to formula (III), preferably in the presence of a catalyst or catalyst system and optionally in the presence of the electrophilic oxidating agent,

wherein R¹ and R² are each independently selected from the group of linear or branched, optionally substituted C₁-C₆ alkyl, alkenyl or alkynyl groups or from the group of optionally substituted C₄-C₁₂ cycloalkyl groups which optionally have one or more unsaturated, exocyclic or endocyclic, carbon carbon bonds or from the group consisting of optionally substituted C₆-C₁₂ aryl groups and optionally substituted C₇-C₁₂ aralkyl groups, e.g. benzyl and 2-phenyl-1-ethyl; R³ is selected from the group consisting of hydrogen, linear or branched C₁-C₆ alkyl groups and C₄-C₁₂ cycloalkyl groups; and PG is a protective group, typically an aryl or benzyl group, preferably selected from the group consisting of the optionally substituted C₆-C₁₄ aryl groups that are preferably activated by electron-donating groups as described above.

Preferably, the reaction mixture of step (A) is not worked-up and step (B) is therefore performed on the reaction mixture resulting from step (A) and in the same reaction vessel in which step (A) is executed.

If substituted, the alkyl, alkenyl, alkynyl, cycloalkyl, aryl and aralkyl groups may be substituted with optionally protected, functional groups comprising one or more heteroatoms such as oxygen, sulphur and nitrogen. Additionally, the alkyl, alkenyl and alkynyl groups may be interrupted by one or more heteroatoms such as oxygen, sulphur and nitrogen, whereas the cycloalkyl, aryl and aralkyl groups may comprise within their ring system one or more heteroatoms such as oxygen, sulphur and nitrogen.

The compounds according to formulas (I)-(III) may occur as enantiomers, diastereomers and mixtures thereof. In particular, the carbon atom to which R¹ is attached and the carbon atom to which R² is attached may have the (R,S)-, (S,R)-, (S,S)- or (R,R)-configuration. If appropriate, the compounds according to formulas (I)-(III) include tautomeric forms.

The present invention furthermore relates to the use of an electrophilic oxidating agent that is optionally formed in situ, said electrophilic oxidating agent being selected from the group of I₂, Br₂, Cl₂ and compounds comprising a halogen atom having a formal oxidation state of 1+, 3+, 5+ or 7+, provided that the electrophilic oxidating agent is not iodobenzene diacetate, in a synthesis of amines, preferably in an asymmetric synthesis of amines.

The invention also relates to all possible combinations of one or more preferred embodiments and/or one or more preferred features as disclosed in this text.

### EXAMPLES

### Example 1

### Synthesis of (2S,3S)-3-(4-methoxyphenylamino)-2-methyl-3-phenylpropan-1-ol (1)

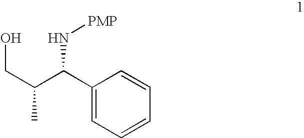

25.0 g (236 mmol) benzaldehyde and 31.9 g p-anisidine (259 mmol) were dissolved in 250 mL NMP. The reaction mixture was stirred for 1.5 h and 2.71 g (23.6 mmol) L-proline was added. The mixture was cooled to −20° C. 51.5 mL (708 mg) propionaldehyde was added dropwise in 30 min. The resulting solution was stirred at −20° C. for 40 h. 250 mL phosphate buffer (50 mM, pH 7) was added. The reaction mixture was allowed to come to ambient temperature and was extracted with 250 mL EtOAc (3×). The combined organic layers were dried and concentrated in vacuo. The residue was taken up in 100 mL methanol and cooled to 0° C. 26.8 g (707 mmol) sodium borohydride was added in portions while the temperature was maintained below −10° C. After complete addition, the reaction mixture was quenched with an aqueous solution of hydrochloric acid until the pH was 1. The reaction mixture was concentrated in vacuo until all methanol had evaporated. The resulting aqueous solution was extracted with 250 mL EtOAc (2×). The combined organic layers were washed with 3×100 mL water, 1×100 mL brine, dried and concentrated in vacuo, which yielded a brown oil. Crystallization from diisopropyl ether yielded 17.7 g (28%) of a white solid 1.

¹H NMR (400 MHz, CDCl₃) δ 0.93 (3H, d, J=7.2 Hz), 2.18 (1H, m), 3.64 (2H, d, J=6.0 Hz), 4.51 (1H, d, J=4.4 Hz), 6.51 (2H, m), 6.68 (2H, m), 7.25 (5H, m)

### Example 2

### Deprotection of 1 with periodic acid under acidic conditions to give (2S,3S)-3-amino-2-methyl-3-phenylpropan-1-ol (2)

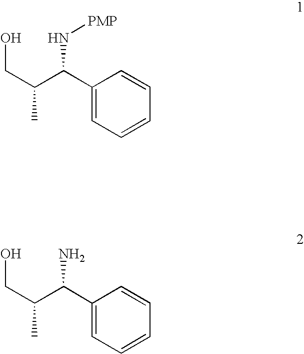

0.30 g (1.1 mmol) 1 was dissolved in 10 mL acetonitrile and subsequently 10 mL water, 0.25 g (1.1 mmol) H₅IO₆, and 1.1 mL 1 M H₂SO₄ were added. The mixture was stirred for 16 h. The reaction mixture was washed with 100 mL CH₂Cl₂ (3×). The resulting aqueous phase was subsequently made alkaline (pH 11) through addition of 5 M KOH solution. The aqueous solution was extracted with 100 mL EtOAc (4×). The combined organic layers were dried over 3 Å molecular sieves and concentrated in vacuo to give pure 2. Yield: 0.14 g (0.84 mmol) (80%) of a colourless oil.

¹H NMR (400 MHz, CD₃OD) δ 0.94 (3H, d, 6.8 Hz), 1.96 (1H, m), 3.31 (1H, dd, J=10.8, J=4.8 Hz), 3.44 (1H, dd, J=10.8 Hz, J=6.4 Hz), 3.93 (1H, d, J=6.0 Hz), 7.28 (5H, m)

### HPLC Method to Follow Deprotection Reaction

| | |
|---|---|
| Column: | Inertsil ODS-3 150 mm × 4.6 mm ID |
| Eluents: | A: H₃PO₄ buffer (10 mM, pH 3.0); B: Acetonitrile |
| Temperature: | 40° C. |
| Flow: | 1.0 mL/min |
| Injection volume: | 5 μL |
| Detection: | UV 210 nm/254 nm |

### Gradient:

| Time (min) | % A | % B |
|---|---|---|
| 0 | 100 | 0 |
| 8 | 25 | 75 |
| 10 | 25 | 75 |
| 12 | 100 | 0 |
| 17 | 100 | 0 |

### Example 3

### Deprotection of N-PMP-(2S,3S)-3-amino-2-methyl-3-phenylpropan-1-ol (1) with various oxidants

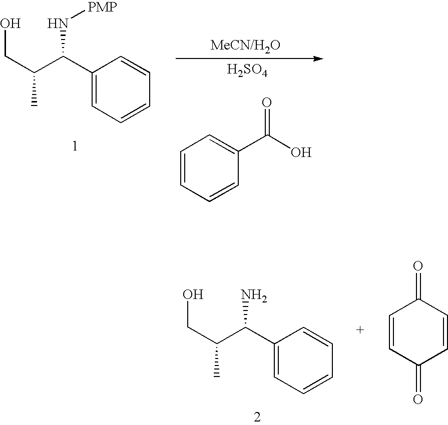

Solutions of 100 mg (0.37 mmol) 1 and 25 mg (0.20 mmol) benzoic acid in 100 ml MeCN/H₂O (1:1) were prepared. Subsequently, 370 μL 1M aqueous H₂SO₄ was added. For each experiment, 10 mL of this solution was transferred to a test tube, containing 1 or 4 equivalents of the oxidant. The resulting solutions were shaken in an orbital shaker at ambient temperature during 8 h and analyzed by HPLC (method described in Example 1). After 20 h, the reaction mixture was analyzed again. The conversion of 1 to 2 was determined using benzoic acid as the internal standard. The results are shown in Table 1.

TABLE 1
Deprotection of N-PMP-protected amines using
various oxidants

| | | | conversion | conversion |
|---|---|---|---|---|
| entry | eq | oxidant | (t = 8 h) [%] | (t = 20 h) [%] |
| 1a | 1 | CAN | 35 | 34 |
| 1b | 4 | CAN | 71 | 42 |
| 2a | 1 | PhI(OAc)₂ | 54 | 47 |
| 2b | 4 | PhI(OAc)₂ | 59 | 64 |
| 3a | 1 | DMI | 47 | 48 |
| 3b | 4 | DMI | 58 | 67 |
| 4a | 1 | TCCA | 79 | 82 |
| 4b | 4 | TCCA | 36 | 0 |
| 5a | 1 | H₅IO₆ | 20 | 81 |
| 5b | 4 | H₅IO₆ | 32 | 75 |
| 6a | 1 | HClO | 40 | 41 |
| 6b | 4 | HClO | 65 | 81 |
| 7a | 1 | PBP | 58 | 60 |
| 8a | 1 | NCS | 41 | 59 |
| 8b | 4 | NCS | 81 | 62 |
| 9a | 1 | NBS | 77 | 79 |
| 9b | 4 | NBS | 75 | 55 |
| 10a | 1 | NIS | 57 | 59 |
| 10b | 4 | NIS | 80 | 78 |
| 11a | 1 | Br₂ | 50 | n.d. |
| 11b | 4 | Br₂ | 50 | n.d. |
| 12a | 1 | I₂ | 53 | 17 |
| 12b | 4 | I₂ | 55 | 21 |

CAN = cerium ammonium nitrate
PhI(OAc)₂ = iodobenzene diacetate
DMI = Dess-Martin Periodinane
TCCA = trichloroisocyanuric acid
PBP = Pyridinium Bromide Perbromide
NCS = N-chlorosuccinimide
NBS = N-bromosuccinimide
NIS = N-iodosuccinimide

It should be noted that CAN and PhI(OAc)₂ are not electrophilic oxidating agent according to the invention, but the two known deprotection agents. A comparison between Entries 1a and b, and 2a and b shows that surprisingly there are many other electrophilic oxidating agents suitable to achieve deprotection of protected amines.

### Comparative Example 1

Experiments (conversion at 8 h) according to the protocol described in Example 3 were also conducted with the inorganic oxidation agents hydrogen peroxide, potassium permanganate, sodium perborate, sodium percarbonate and potassium bichromate. However, the deprotection reaction did not proceed or only produced very low yields and considerable amounts of side products.

### Example 4

### Preparative Experiments

### Deprotection of (2S,3S)-3-((4-methoxyphenyl)(methyl)amino)-2-methyl-3-(4-nitrophenyl)propan-1-ol (3)

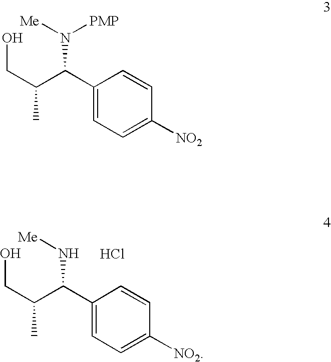

0.50 g (1.5 mmol) 3 was dissolved in 30 mL acetonitrile and subsequently 30 mL water, 0.18 g (0.76 mmol) TCCA, and 1.5 mL 1 M H₂SO₄ were added. The mixture was heated to 90° C. and stirred for 30 min. The reaction mixture was allowed to come to ambient temperature and was washed with 50 mL CH₂Cl₂ (3×). The resulting aqueous phase was subsequently made alkaline (pH 11) through addition of 5 M KOH solution. The aqueous solution was extracted with 50 mL EtOAc (3×). The combined organic layers were dried over 3 Å molecular sieves and concentrated in vacuo. The residue was dissolved in 50 mL EtOAc and 10 mL of a saturated solution of hydrochloric acid in EtOAc was added. Concentration in vacuo yielded 0.31 g of pure 4 (1.2 mmol) (77%) as a white powder.

¹H NMR (400 MHz, MeOH-d4) δ 0.88 (3H, d, J=7.2 Hz), 2.53 (1H, m), 2.55 (3H, s), 3.27 (1H, dd, J=10.8 Hz, J=9.2 Hz), 3.54 (1H, dd, J=4.4 Hz, J=10.8 Hz), 4.22 (1H, d, J=4.4 Hz), 7.72 (2H, m), 8.35 (2H, m)

### Deprotection of 4-methoxy-N-(1-phenylethyl)aniline (5)

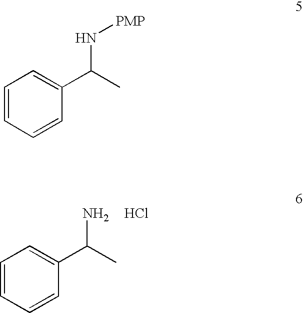

0.50 g (2.2 mmol; racemic) 5 was dissolved in 20 mL acetonitrile and subsequently 20 mL water, 0.25 g (1.1 mmol) TCCA, and 2.2 mL 1 M H₂SO₄ were added. The mixture was stirred for 16 h. The reaction mixture was allowed to come to ambient temperature and was washed with 50 mL CH₂Cl₂ (3×). The resulting aqueous phase was subsequently made alkaline (pH 11) through addition of 5 M KOH solution. The aqueous solution was extracted with 50 mL EtOAc (3×). The combined organic layers were dried over 3 Å molecular sieves and concentrated in vacuo. The residue was dissolved in 50 mL EtOAc and 10 mL of a saturated solution of hydrochloric acid in EtOAc was added. Concentration in vacuo yielded 0.35 g of pure 6 (2.2 mmol) (99%) as an off-white powder.

¹H NMR (400 MHz, MeOH-d₄) δ 1.63 (3H, d, J=6.8 Hz), 3.29 (1H, m), 4.45 (1H, q, J=6.8 Hz), 7.42 (5H, m)

### Example 5

### Synthesis of (2S,3S)-3-amino-2-methyl-3-phenylpropanoic acid (7)

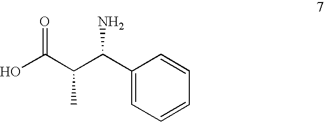

0.50 g (1.9 mmol) of 1 was dissolved in MeCN/H₂O (20 mL, 1:1). 1.9 mL 1M H₂SO₄ was added, followed by 0.42 g (1.9 mmol) H₅IO₆. The reaction mixture was stirred at ambient temperature for 16 h. Subsequently, 27 mg Na₂Cr₂O₇.2H₂O (0.092 mmol) was added, followed by 2.1 g (0.92 mmol) H₅IO₆. The mixture was stirred at ambient temperature for 16 h. After completion, the reaction mixture was washed with 50 mL CH₂Cl₂ (3×) and purified by ion exchange chromatography. Amino acid 7 was obtained as an off-white solid.

C₁₀H₁₃NO₂, 179.22 g/mol

¹H-NMR (300 MHz, D₂O) δ 1.27 (d, 3H, J=6.8 Hz), 3.01 (m, 1H), 4.47 (d, 1H, J=8.1 Hz), 7.47 (m, 5H)

¹³C-NMR (75 MHz, D₂O) δ 13.5, 45.4, 57.1, 126.7, 128.6, 128.8, 134.8, 179.5

IR v (cm⁻¹): 2972, 1559, 1454, 1401, 1368, 1208, 1107, 1065, 1032, 755, 700

HRMS (ESI+) calcd for C₁₀H₁₂NO₂Na₂ 224.0663. Found: 224.0666

### Example 6

### Synthesis of (2S,3S)-3-amino-2-methyl-3-(4-nitrophenyl)propanoic acid (9)

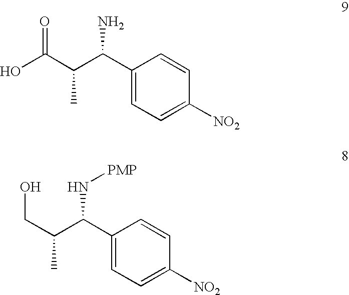

Compound 9 was prepared from compound 8 according to the method of Example 5.

C₁₀H₁₂N₂O₄, 224.21 g/mol. Yellow solid.

¹H-NMR (300 MHz, D₂O) δ 1.27 (d, 3H, J=6.9 Hz), 2.98 (m, 1H), 4.59 (d, 1H, J=8.1 Hz), 7.66 (d, 2H, J=8.4 Hz), 8.33 (d, 2H, J=8.4 Hz)

¹³C-NMR (75 MHz, D₂O) δ 179.4, 147.4, 142.2, 127.980, 123.7, 56.5, 45.7, 13.4

IR v (cm⁻¹): 2969, 1737, 1365, 1229, 1216, 735, 668, 611

HRMS (ESI+) calcd for C₁₀H₁₂N₂O₄Na 247.0695. Found: 247.0716

1-23. (canceled)

24. A process for the deprotection of protected amine compounds, wherein the protected amine compound is contacted with an electrophilic oxidating agent that is optionally formed in situ, said electrophilic oxidating agent being selected from the group of 12, Br₂, Cl₂ and compounds comprising a halogen atom having a formal oxidation state of 1+, 3+, 5+ or 7+, provided that the electrophilic oxidating agent is not iodobenzene diacetate.

25. The process according to claim 24, wherein the halogen atom has a formal oxidation state of 1+, 5+ or 7+.

26. The process according to claim 25, wherein the halogen atom has a formal oxidation state of 1+.

27. The process according to claim 24, wherein the electrophilic oxidating agent is an organic electrophilic oxidation agent.

28. The process according to claim 24, wherein the process is conducted at acidic pH.

29. The process according to claim 24, wherein the protected amine compound is a compound comprising a secondary amine.

30. The process according to claim 24, wherein the amino group of the protected amine compound is protected by an optionally substituted C₆-C₁₄ aryl group.

31. The process according to claim 30, wherein the aryl group is substituted with an electron-donating group.

32. The process according to claim 30, wherein the aryl group is a phenyl group.

33. The process according to claim 32, wherein the phenyl group is substituted with one or more an electron-donating group(s).

34. The process according to claim 32, wherein the phenyl group is substituted on an ortho-position and/or the para-position.

35. A process for the asymmetric synthesis of amines, said process comprising the steps of:
a. reacting a carbonyl compound with an amine compound that is at least substituted by an aryl group, preferably by a C₆-C₁₄ aryl group under formation of a protected imine compound;
b. converting the protected imine compound into a protected amine compound; and
c. deprotecting the protected amine compound according to the process according to claim 24.

36. The process according to claim 35, wherein the carbonyl compound is a ketone, an aldehyde, a protected ketone, a protected aldehyde or a precursor of a ketone or an aldehyde.

37. The process according to claim 35, wherein the amine is selected from the group consisting of 1,3-amino alcohols, β-amino acids and heterocyclic compounds.

38. Method for the preparation of F-amino acids comprising the steps of: 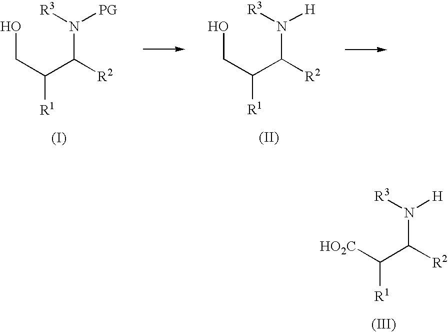
(A) deprotecting a protected amine compound according to formula (I) by contacting said protected amine compound with an electrophilic oxidating agent that is optionally formed in situ, said electrophilic oxidating agent being selected from the group of I₂, Br₂, Cl₂ and compounds comprising a halogen atom having a formal oxidation state of 1+, 3+, 5+ or 7+ to a deprotected amine compound according to formula (II); and
(B) oxidising the deprotected amine compound according to formula (II) to β-amino acid according to formula (III),
wherein:
R1 is selected from the group of linear or branched, optionally substituted C1-C6 alkyl, alkenyl or alkynyl groups and optionally substituted C₄-C₁₂ cycloalkyl groups which optionally have one or more unsaturated, exocyclic or endocyclic, carbon carbon bonds;
R₂ is selected from the group consisting of optionally substituted C₆-C₁₂ aryl groups and optionally substituted C₇-C₁₂ aralkyl groups;
R₃ is selected from the group consisting of hydrogen, linear or branched C₁-C₆ alkyl groups and C₄-C₁₂ cycloalkyl groups; and
PG is a protective group selected from the group of aryl or benzyl groups, preferably selected from the group consisting of optionally substituted C₆-C₁₄ aryl groups.
